(19) United States
(12) Patent Application Publication
(10) Pub. No.: US 2009/0013444 A1
(43) Pub. Date: Jan. 15, 2009

(54) CLOTHING

(75) Inventors: Atsuko Misaki, Kyoto (JP); Tatsuzo Nakamura, Osaka (JP)

Correspondence Address: GREENBLUM & BERNSTEIN, P.L.C., 1950 ROLAND CLARKE PLACE, RESTON, VA, 20191, US

(73) Assignee: WACOAL CORP., Kyoto (JP)

(21) Appl. No.: 11/909,739

(22) PCT Filed: Mar. 14, 2006
(86) PCT No.: PCT/JP2006/305002

§ 371 Date: Sep. 26, 2007

(30) Foreign Application Priority Data
Mar. 28, 2005 (JP) 2005-092301

(51) Int. Cl. A41D 1/00; A41C 3/00; A61B 17/00

(52) U.S. Cl. 2/69; 450/1; 606/204

(57) ABSTRACT

Clothing provided, on the inner surface side thereof at such a position as covering the meridian point of a human body wearing the clothing, with a plurality of elastic small protrusions having a diameter of 2-4 mm at the bottom face thereof and located in a region of 50-120 mm² for one meridian point. Even if the positional shift takes place of the clothing and regardless of difference in physique of a wearing object, any one of the small elastic protrusions is positioned oppositely to the meridian point and imparts a slight tactile stimulus to meridian points P1-P3.

### TECHNICAL FIELD

The present invention relates to clothing which imparts a light tactile stimulus to meridian points (so-called therapeutic vital pressure points) of a human body to accelerate the circulation of blood so that the stiffness of the shoulders, cold, menorrhalgia, and lumbago can be softened.

### BACKGROUND ART

Many persons suffer from the stiffness of the shoulders. There is a report of investigation that about 60% of women having a desk work chronically feel stiff in their shoulders and about 30% thereof sometimes feel stiff in their shoulders (City Living Report n=868).

As methods of softening the stiffness of the shoulders, treatment by stimulating pressure points of a human body, massage, moxa cautery, acupuncture, magnetic treatment, and the like are known. But in many cases, to soften the stiffness of the shoulders by these treating methods, it is necessary for a person suffering the stiffness of the shoulders to go to infirmaries. Even when treatment for softening the stiffness of the shoulders is made at home, it is necessary to prepare a treating appliance at home, and the treatment requires a lot of time. Thus it is difficult to carry out the above-described treating methods at home when the person leads a busy life. When the degree of the stiffness of the shoulders is low, there is no need for treatment. But if the stiffness of the shoulders is chronic, the stiffness of the shoulders is liable to become serious.

Therefore it is very useful to provide a shoulder strap or other portions of clothing with a devise which is capable of softening the stiffness of the shoulders and eliminates the need for the treatment of softening the stiffness of the shoulders.

Recently owing to a decrease in the function of their autonomic nerves, poverty of blood or low blood pressure, women have a decrease or stagnation in the circulation of blood. Consequently not only cold but also menorrhalgia and lumbago are liable to be generated on the periphery of the pelvis.

Therefore by accelerating the circulation of blood on the periphery of the pelvis, it is possible to effectively soften the cold, the menorrhalgia, and the lumbago. To accelerate the circulation of blood, it is said that to stimulate the meridian points is effective. Thus by stimulating the meridian points, it is possible to accelerate the circulation of blood and soften the cold, the menorrhalgia, and the lumbago.

From the above-described standpoint, in Japanese Patent Application Laid-Open No. 11-61509 (patent document 1), there is proposed a brassiere having a magnet mounted on the shoulder strap thereof. Description is made in the specification that the stiffness of the shoulders of a person wearing the brassiere can be improved by the magnetism of the magnet mounted on the shoulder strap.

But because the magnet is embedded inside the shoulder strap, many persons do not feel the effect of the magnetism.

As meridian points present in the region where the stiffness of the shoulders is generated, kensei, tenryou, and kengaiyu are known. But the shoulder strap of normal clothing such as the brassiere is disposed outward from the meridian points not over the meridian points when the clothing is worn. Thus it is impossible to apply the magnetism to the meridian points by pressing the magnet mounted on the shoulder strap against the meridian points. This is true of a case in which a means for decreasing the degree of the stiffness of the shoulders other than the magnet is mounted on the shoulder strap.

In Japanese Patent Application Laid-Open No. 10-24085 (patent document 2), there is proposed a “pad to be pressed against a pressure point for softening stiffness” which can be pressed against a desired pressure point for softening stiffness. The “pad to be pressed against a pressure point for softening stiffness” has a protrusion. With the protrusion applied to a desired pressure point for softening stiffness to be pressed, the “pad to be pressed against a pressure point for softening stiffness” is mounted on a human body by adhesion or with the protrusion located at a position on the inner surface of clothing corresponding to the desired pressure point for softening stiffness, the “pad to be pressed against a pressure point for softening stiffness” is mounted on the clothing.

In mounting the “pad to be pressed against a pressure point for softening stiffness” on the human body, it is necessary to re-mount it on the human body every day. Thus it is necessary to look for the pressure point for softening stiffness and stick the “pad to be pressed against a pressure point for softening stiffness” thereto each time the stiffness of the shoulders is softened, which takes time and trouble. In mounting the “pad to be pressed against a pressure point for softening stiffness” on the inner surface of the clothing, the protrusion cannot be securely applied to the pressure point for softening stiffness when the clothing is dislocated from the human body.

In Japanese Utility Model Application Laid-Open No. 54-149407 (patent document 3), there is proposed clothes in which a portion of the back panel having a wide region covering the meridian points at the back side of the human body has a large thickness.

Because the thick portion of the clothes proposed in the patent document 3 is wide, the thick portion cannot press the desired meridian point locally with a pinpoint accuracy, but presses a wide region of the back side of the human body. Thereby the clothes have a problem that a person wears the clothes uncomfortably and is thickly clad.

Patent document 1: Japanese Patent Application Laid-Open No. 11-61509

Patent document 2: Japanese Patent Application Laid-Open No. 10-24085

Patent document 3: Japanese Utility Model Application Laid-Open No. 54-149407

### DISCLOSURE OF THE INVENTION

### Problem to be Solved by the Invention

The present invention has been made in view of the above-described problems. It is an object of the present invention to provide clothing which eliminates the need for going to a chiropractor or a massager and the use of a massaging tool or the like and is capable of continually imparting a light tactile stimulus to meridian points to such an extent that the clothing touches the meridian points and imparting a little strong pressing force to the meridian points by a person's ordinary motion of moving the body thereof to thereby accelerate the circulation of blood and soften the stiffness of the shoulders, cold and menorrhalgia generated in the waist and the belly, and lumbago, by only wearing the clothing.

### Means for Solving the Problem

To solve the above-described problems, the present invention provides clothing including a plurality of elastic small protrusions, each having a diameter of 2 to 4 mm at a bottom face thereof, which are provided on an inner surface thereof at positions covering meridian points of a human body wearing the clothing, with the elastic small protrusions disposed in a region of 50 to 120 mm² for one of the meridian points so that even if a positional shift of the clothing takes place and irrespective of a physical constitution of the human body wearing the clothing, any one of the elastic small protrusions is disposed at a position where the elastic small protrusion confronts one of the meridian points and imparts a tactile stimulus to the meridian point.

The meridian point is a point, most sensitive to perception, which is present on a vigor-blood transmission path and through which an organic reaction appears greatly on the skin. The meridian point is coincident with various hypersensitive points, tenderness points, and linkage points. In acupunture and moxibustion clinical medicine, the meridian point is stimulated to accelerate the circulation of blood so that the stiffness of the shoulders, cold, menorrhalgia, and lumbago can be softened.

More specifically, as the meridian points (pressure points for softening stiffness) which are capable of softening stiffness on the periphery of the upper region of the shoulders, as shown in FIGS. 9(A), 9(B), kensei P1, tenryou P2, and kengaiyu (P3) are known. The kensei P1 is disposed at approximately the mid-position of a line connecting the kenketsu and the daitsuiketsu. The tenryou P2 is disposed at the mid-position, between the kenseiketsu and the kyokugakiketsu, which is located outward and upward from the angulus superior scapulae. The kengaiyu P3 is disposed outward (both sides) by an interval of 3 sun (1 sun=3.03 cm) from the mid-position, between the first thoracic spine acantha and the second thoracic spine acantha, which is located on the periphery of the angulus superior scapulae.

As the meridian points, disposed on the periphery of the back-side part of the shoulders, which are capable of softening the stiffness of the shoulders, koukou P4 and kanyu P5 are known. The koukou P4 is disposed outward (both sides) by an interval of 3 sun from the mid-position between the fourth thoracic spine acantha and the fifth thoracic spine acantha. The kanyu P5 is disposed outward (both sides) by an interval of 1 sun 5 bu (1 bu=0.119 inches) from the mid-position between the ninth thoracic spine acantha and the tenth thoracic spine acantha.

As the meridian points, disposed at the belly, which are capable of softening the cold, the menorrhalgia, and the lumbago, as shown in FIGS. 10(A) and 10(B), daikyo P6, kikai P7, kangen P8, and chukyoku P9 are known.

As the meridian points disposed at the waist, daichoyu P10, jiryou P11, churyou P12, houkou P13, jinyu P14, and jousen P15 are known.

The clothing of the present invention does not impart a stimulus equivalent to that imparted by acupuncture and moxa cautery treatment to the meridian point to soften the stiffness of the shoulders, the cold, the menorrhalgia, and the lumbago. In the clothing of the present invention, the elastic small protrusions lightly contact the meridian points respectively, thus constantly imparting a tactile stimulus to the meridian points. Further the elastic small protrusions are pressed temporarily and a little strongly against the meridian points respectively by a person's motion or the person's simple motion of pressing the elastic small protrusions lightly with fingers to impart a temporary and light stimulus. Thereby the meridian points are stimulated to accelerate the circulation of blood so that it is possible to soften stiffness such as the stiffness of the shoulders, the cold, the menorrhalgia, and the lumbago.

In the present invention, as described above, it is preferable to provide the clothing with a plurality of the elastic small protrusions or preferably four elastic small protrusions inside the region having the area of 50 mm² to 120 mm² at the position covering one meridian point with the four elastic small protrusions spaced at intervals of 90 degrees. When the four elastic small protrusions are disposed at a high density with the four elastic small protrusions spaced at the intervals of 90 degrees as described above, it is possible to securely place any one of the four elastic small protrusions at a position where it confronts one of the meridian points when the person wears the clothing or moves the person's body, even though the elastic small protrusions are dislocated from the predetermined positions in a vertical direction or a horizontal direction. If the clothing is provided with a protrusion having a large area instead of the above-described small protrusion, the large protrusion widely contacts not only the meridian points but also the periphery thereof. Thus there is a decrease in the function of imparting a stimulus to the meridian points with a pinpoint accuracy.

Three to five elastic small protrusions may be provided in the region having the area of 50 mm² to 120 mm². But according to the present inventors' experiments, it has been found that most favorably four elastic small protrusions are provided inside the region having the area of 50 mm² to 120 mm² because any one of the four elastic small protrusions can be placed at one of the meridian points even though the elastic small protrusions are dislocated from the predetermined positions in the vertical direction or the horizontal direction.

The elastic small protrusions may be disposed regularly by forming one group of the four elastic small protrusions. For example, the elastic small protrusions may be provided in the longitudinal direction of the shoulder strap of the clothing or/and a plurality of groups of the elastic small protrusions may be provided by successively arranging them in line.

In the above-described construction, by stimulating a plurality of the meridian points by the elastic small protrusions located at a plurality of the meridian points proximate to each other, it is possible to soften the stiffness of the shoulders, the cold, the menorrhalgia, and the lumbago.

As a plurality of the meridian points proximate to each other, for example, the above-described kensei P1, tenryou P2, and kengaiyu P3 capable of softening the stiffness of the shoulders are known.

Thus in covering the three meridian points, namely, the kensei P1, the tenryou P2, and the kengaiyu P3, the region in which the elastic small protrusions are disposed is set to 10 to 20 mm in its width and 50 to 120 mm, favorably 70 to 100 mm, and more favorably 90 mm in its length.

It is preferable to set the height of the elastic small protrusions to the range of 2 to 4 mm.

The reason the height of the elastic small protrusions is set to the range of 2 to 4 mm is because the elastic small protrusions having the height in the above-described range do not make the person feel uncomfortable, when the elastic small protrusions impart the tactile stimulus to the person with the elastic small protrusions in contact with the person and because the person does not feel a pain even though the elastic small protrusions are temporarily pressed against the person at a little strong force when the person moves her/his body.

When a base material on which the elastic small protrusions are provided is made of a material having a high tension, the height of the elastic small protrusions is set small in the range, whereas when the base material on which the elastic small protrusions are provided is made of a material having a low tension, the height of the elastic small protrusions is set large in the range.

The reason the height of the elastic small protrusions is adjusted according to the kind of the base material on which the elastic small protrusions are mounted is as follows: When the base material is made of the material having a low tension, the portion of the base material on which the elastic small protrusions are mounted expands outward and is pressed against the skin in a small height. On the other hand, when the base material is made of the material having a high tension, the portion on which the elastic small protrusions are mounted does not expand outward and is pressed against the skin in a large height. Therefore it is preferable to change the height of the elastic small protrusions according to the kind of the base material on which the elastic small protrusions are mounted so that the elastic small protrusions stimulate the human body at almost a constant force. It is preferable to set the difference between the height of the elastic small protrusions mounted on the material having a high tension and that of the elastic small protrusions mounted on the material having a low tension to about 1 mm.

It is preferable that the elastic small protrusions are made of resin or fiber and that the difference between the height of the elastic small protrusions when no load is applied thereto and the height thereof when a load of 50 g is applied thereto is set to less than 1 mm.

The reason the above-described difference in the height of the elastic small protrusions according to the load applied thereto is set to less than 1 mm is because if the height difference is more than 1 mm, the elastic small protrusions are deformed when a small load is applied thereto and the elastic small protrusions are affected more than the person's skin. Consequently when the elastic small protrusions are pressed against the meridian points, the elastic small protrusions are incapable of sufficiently stimulating the meridian point, thus having little effect of accelerating the circulation of blood.

The difference between the height of the elastic small protrusion when no load is applied thereto and the height thereof when the load of 50 g is applied thereto is set to less than favorably 1 mm and more favorably less than 0.7 mm.

In making the elastic small protrusions of resin, the elastic small protrusions are made of silicone, for example, and are fixed to necessary positions. In making the elastic small protrusions of fiber, the elastic small protrusions are formed by intertwining threads with one another or by using a knitted structure or a woven structure.

It is preferable that each of the elastic small protrusions is semispherical, spherical or a conic configuration, a leading portion of which is formed as a circular-arc surface.

When the elastic small protrusions have any one of the above-described configurations, the elastic small protrusions are hardly deformed and capable of holding a proper degree of hardness.

The base material on which the elastic small protrusions are provided is made of an unstretchable material or a difficult-to-stretch material; the base material is fixedly mounted on the clothing body at a necessary position thereof or removably mounted on a holding portion provided on the clothing body at a necessary position thereof, with the elastic small protrusions protruded toward the skin of the human body.

It is possible to fix the base material, made of the unstretchable material or the difficult-to-stretch material, on which the elastic small protrusions are mounted to the clothing body by sewing the base material thereto or it is possible to removably mount the base material having the elastic small protrusions mounted thereon on the clothing body. When the base material having the elastic small protrusions mounted thereon is removably mounted on the clothing body, it is possible to prevent the elastic small protrusions from being crushed or peeled off the base material by removing the base material from the clothing body when the clothing is washed. As methods of removably mounting the base material on the clothing body, it is possible to provide the clothing body with a bag portion having an opening from which both ends of the base material are inserted or provide the clothing body with a face fastener which is locked to the rear surface of the base material and the clothing body.

It is preferable that the region in which the elastic small protrusions are provided is covered with a covering cloth consisting of thin cloth so that the elastic small protrusions are brought into contact with the skin of the human body through the covering cloth.

In this construction, because the elastic small protrusions do not directly contact the person's skin, it is possible to decrease the degree to which marks are generated on the person's skin by the elastic small protrusions and prevent the skin from being roughened, even though the person wears the clothing of the present invention for a long time.

In removably mounting the base material on which the elastic small protrusions have been mounted on the clothing, it is preferable to mount the covering cloth on the clothing, form a bag-shaped portion by the clothing body and the covering cloth, and insert the base material on which the elastic small protrusions have been mounted into the bag-shaped portion from an opening formed at one end thereof.

As a measure for softening the stiffness of the shoulders, specifically, a wide base material made of an unstretchable material or a difficult-to-stretch material is attached to the left and right shoulder strap bodies mounted on the clothing from the front panel thereof to the back panel thereof at positions thereof essentially covering the meridian points disposed on a periphery of an upper region of a shoulder, with the wide base material protruded toward the neck of the human body; and the wide base material is provided with the elastic small protrusions located at positions of an inner surface thereof covering the meridian points.

As described above, when the clothing having the shoulder strap is a brassiere, the shoulder strap does not pass the meridian points, but a region outward (side opposite to neck) from the meridian points. Therefore even though the shoulder strap is provided with the elastic small protrusions, it is impossible to place the elastic small protrusions at positions where they confront the meridian points.

On the other hand, the shoulder strap body is provided with the wide belt part protruded toward the neck as described above with the wide belt part placed over the meridian points. Thus by providing the wide belt part with the elastic small protrusions, the elastic small protrusions are capable of stimulating the meridian points.

Alternatively, the left and right shoulder strap bodies mounted on the clothing from the front panel thereof to the back panel thereof are connected with each other with the left and right shoulder strap bodies disposed in proximity to each other at a front panel side and a back panel side; the left and right shoulder strap bodies are brought near to the neck in an upper region of a shoulder so that the left and right shoulder strap bodies cover the meridian points; and each of the left and right shoulder strap bodies is widened in the upper region of the shoulders and made of a unstretchable material or a difficult-to-stretch material. Each of the left and right shoulder strap bodies widened in the upper region of the shoulders is provided with the elastic small protrusions by locating the elastic small protrusions at meridian point-covering positions of an inner surface of the left and right shoulder strap bodies.

As described above, the left and right shoulder strap bodies are connected with each other with left and right the shoulder strap bodies disposed in proximity to each other at the front panel side and the back panel side. Further the left and right shoulder strap bodies are brought near to the neck in the upper region of the shoulder so that the left and right shoulder strap bodies cover the meridian points. Furthermore each of the left and right shoulder strap bodies is widened in the upper region of the shoulder. Therefore by mounting the elastic small protrusions on the shoulder strap bodies, it is possible to locate the elastic small protrusions at positions where they contact the meridian points.

In above-described both constructions, the wide belt part of the shoulder strap or the shoulder strap body on which the elastic small protrusions are mounted is made of the unstretchable material or the difficult-to-stretch material so that the wide belt part or the shoulder strap body does not extend or little extends in the longitudinal direction thereof. Therefore it is possible to prevent the elastic small protrusions from being dislocated in the longitudinal direction of the wide belt part or the shoulder strap body or from departing from the meridian points irrespective of a person's physical constitution.

The elastic small protrusions are mounted on the unstretchable material or the difficult-to-stretch material so that the elastic small protrusions are hardly dislocated. Further the elastic small protrusions are densely disposed thereon. Thereby irrespective of dislocation of the elastic small protrusions to some extent or irrespective of the person's physical constitution, the elastic small protrusions can be securely located at the positions where they confront the meridian points.

As the unstretchable material and the difficult-to-stretch material, cotton, nylon, and polyester are listed.

As a measure for softening the “the stiffness of the shoulders”, the elastic small protrusions are mounted on the inner surface of the clothing at positions covering the meridian points positioned on the person's back, for example, the koukou P4 and the kanyu P5.

That is, it is preferable that on the back panel continuous with the shoulder strap, a plurality of the elastic small protrusions or preferably four elastic small protrusions are provided at positions where the elastic small protrusions cover the meridian points with the elastic small protrusions disposed in the region of 50 to 120 mm² for one of the meridian points. Thereby even though the back panel is dislocated from the person's body, one elastic small protrusion can be located at the position where the elastic small protrusion confronts the meridian point with a pinpoint accuracy.

To soften the cold, the menorrhalgia, and the lumbago, the elastic small protrusions are mounted on the inner surface of clothing, for example, shorts, girdle, and the like at positions thereof covering the meridian points of the belly, namely, the daikyo P6, the kikai P7, the kaigen P8, the chukyoku P9 or/and the meridian points of the waist, namely, the daichoyu P10, the jiryou P11, the churyou P12, and houkou P13.

By providing the shorts and the girdle with the elastic small protrusions as described above, the elastic small protrusions always impart the tactile stimulus to such an extent that the elastic small protrusions lightly touch the meridian points respectively by only wearing the shorts or/and the girdle. Thus the person does not have a feeling of physical disorder. Further the height of the elastic small protrusions is set as small as 2 to 4 mm. Thus marks of the elastic small protrusions are little left on the skin of the belly and the waist. Further the elastic small protrusions do not adversely affect an outerwear in the appearance thereof. The shorts and the girdle provided with the elastic small protrusions do not have an outlook different from that of shorts and girdles not provided with the elastic small protrusions provided thereon.

The present invention is applicable to clothing for women and men which directly contacts the person's skin and clothing for women and men which is worn on thin underwear, provided that they cover the meridian points. More specifically, it is possible to list underwear such as a brassiere, a brassiere-provided slip, lingerie, undershirt, girdle, shorts, pants; and sports clothing such as a swimming suit, leotard, and the like.

### EFFECT OF THE INVENTION

As described above, according to the present invention, because the elastic small protrusions are densely arranged in the positions covering the meridian points, any one of the elastic small protrusions can be disposed at the position where it confronts one of the meridian points, even though the clothing is dislocated from a human body and irrespective of the physical constitution thereof. The elastic small protrusion confronting the meridian point is capable of imparting a weak tactile stimulus to the meridian point with the elastic small protrusion in contact with the meridian point at a weak force to such an extent that the elastic small protrusion continually touch the meridian point.

By a person's simple motion of lightly pressing the elastic small protrusions with fingers or raising the shoulders, it is possible to temporarily impart a little strong stimulus to the meridian points at any time by the elastic small protrusions and make the person temporarily comfortable.

Further by constantly impart the tactile stimulus to the meridian points with the elastic small protrusions disposed at the meridian points capable of softening the cold, the menorrhalgia, and the lumbago and by temporarily pressing the meridian points with the elastic small protrusions to stimulate them, it is possible to accelerate the circulation of blood and soften the cold, the menorrhalgia, and the lumbago.

The elastic small protrusions are mounted on the unstretchable material or the difficult-to-stretch material to prevent the elastic small protrusions from being dislocated from the predetermined positions. Further the elastic small protrusions are densely arranged. Thereby even though the elastic small protrusions are dislocated from the predetermined positions and irrespective of a person's physical constitution, it is possible to securely place the elastic small protrusions at the meridian points respectively.

### BRIEF DESCRIPTION OF THE DRAWINGS

FIG. 1 is a perspective view showing a brassiere of a first embodiment of the present invention.

FIG. 2(A) shows a wide part of a shoulder strap; 2(B) is a main part-enlarged view showing arrangement of elastic small protrusions; and 2(C) is a sectional view taken along a line A-A of FIG. 2(A).

FIGS. 3(A), 3(B) show a modification of the first embodiment.

FIG. 4 is a perspective view showing a brassiere of a second embodiment.

FIGS. 5(A), 5(B) show a modification of the second embodiment.

FIG. 6 is a perspective view showing a brassiere of a third embodiment.

FIGS. 7(A) and (B) show a girdle of a fourth embodiment.

FIG. 8 is a sectional view taken along a line X-X of FIG. 7(A).

FIGS. 9(A) and (B) show meridian points (pressure point for softening stiffness) capable of softening stiffness on the periphery of the shoulder.

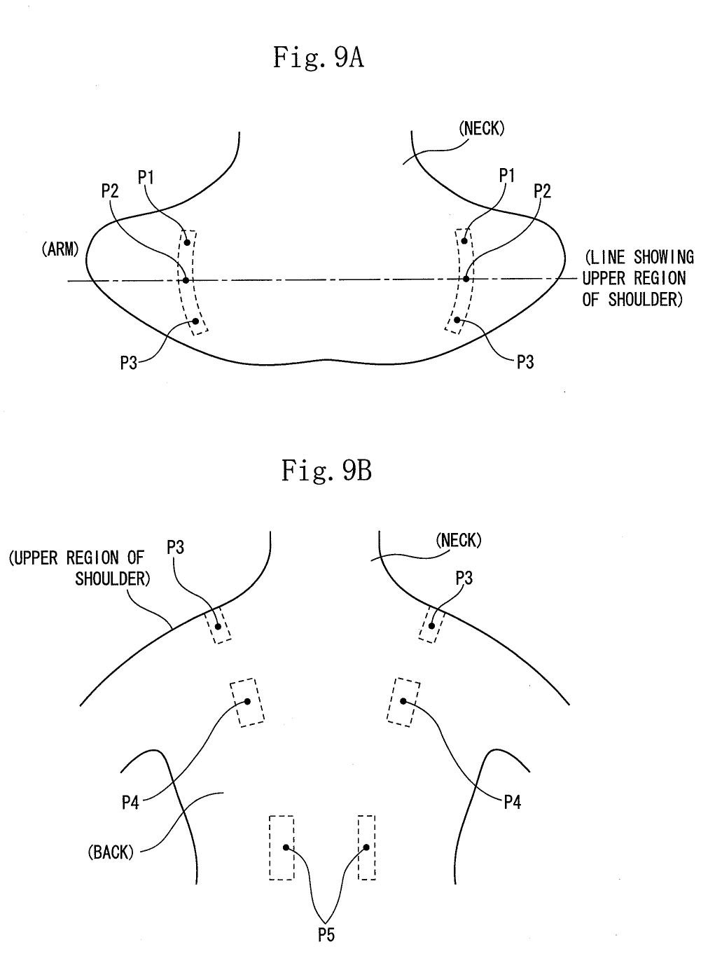

FIG. 10(A) shows meridian points (pressure point for softening stiffness) capable of softening cold of the belly; and FIG. 10(B) shows meridian points (pressure point for softening stiffness) capable of softening cold of the waist.

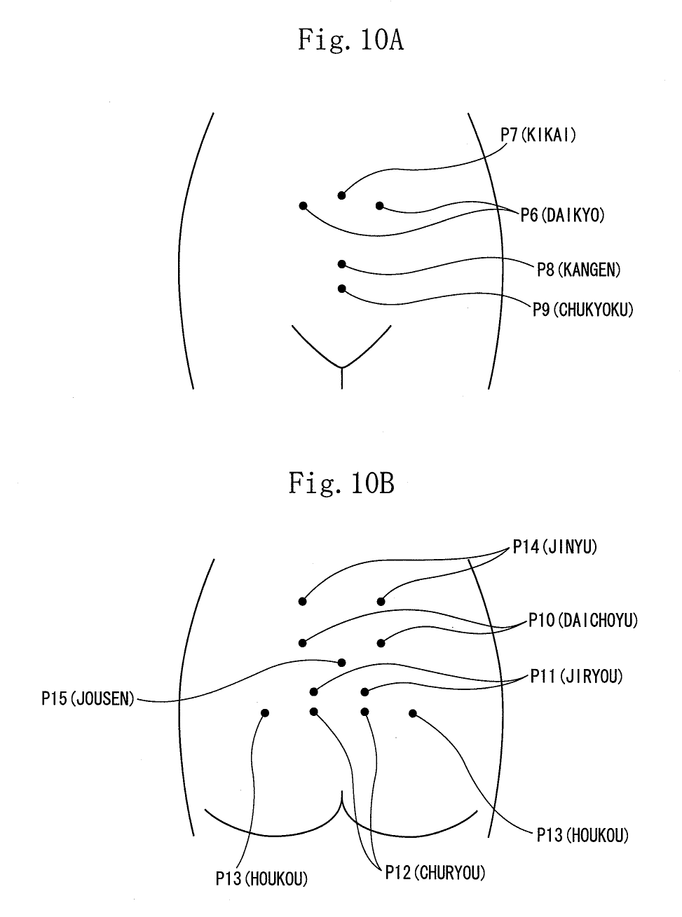

### EXPLANATION OF REFERENCE NUMERALS AND SYMBOLS

10: brassiere:
11: cup cloth
14: shoulder strap
14a: shoulder strap body
15: wide belt part
16: covering cloth
20: elastic small protrusion
P1 through P13: meridian points (pressure point for softening stiffness)
40: girdle

### BEST MODE FOR CARRYING OUT THE INVENTION

The embodiments of the present invention are described below with reference to the drawings.

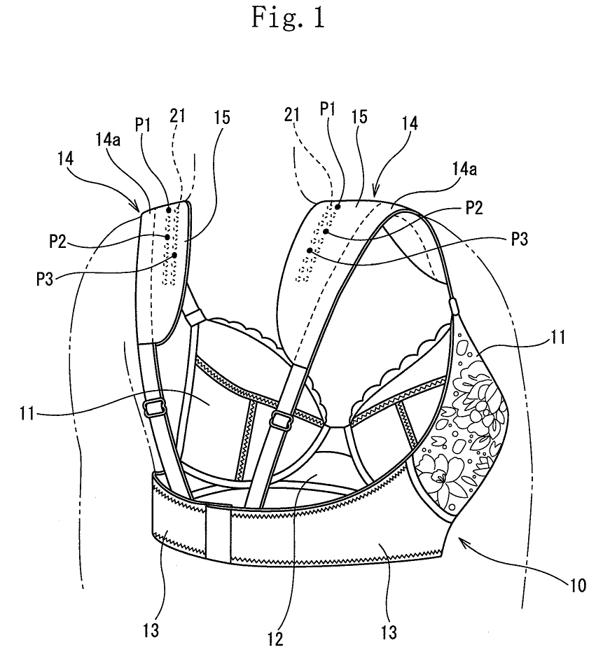

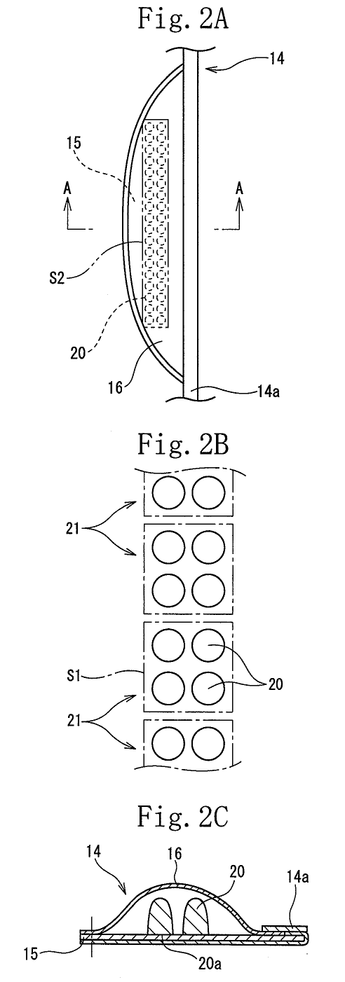

FIGS. 1 and 2 show the first embodiment of the present invention. Clothing of the first embodiment having shoulder straps consists of a brassiere 10. The brassiere 10 includes left and right cup cloths 11 forming a front panel; a supporting belt cloth 12 sewed to a lower edge of an inner side of the left and right cup cloths 11; left and right back parts 13, forming a back panel, which are sewed to a lower edge of left and right outer sides of the left and right cup cloths 11 respectively; and left and right shoulder straps 14 mounted at an upper end of the left and right cup cloths 11 respectively and an upper end of the back part 13. Each of the left and right back parts 13 is provided with a fastener (not shown) disposed at a tip of a rear surface thereof. The fasteners are locked to each other.

A position where the shoulder strap 14 and the left and right cup cloths 11 are sewed to each other and a position where the shoulder strap 14 and the back part 13 are sewed to each other are located inward by 1 cm respectively from sewing positions in ordinary brassieres. A wide belt part 15 made of a base material such as cotton, nylon or polyester which is an unstretchable material or a difficult-to-stretch material is attached to a shoulder strap body 14a of the shoulder strap 14 by sewing the wide belt part 15 to the shoulder strap body 14a at a position covering the meridian points disposed on the periphery of the upper region of the shoulder with the wide belt part 15 protruded toward the neck.

As shown in FIG. 2(B), four elastic small protrusions 20 are welded to a skin side of the wide belt part 15, namely, an inner surface of the wide belt part 15 mounted on the shoulder strap body 14a of the left and right shoulder straps 14 at a 10-millimeter square region of the inner surface thereof, namely, a region S1 having an area of 100 mm² with the elastic small protrusions 20 spaced at equal intervals vertically and horizontally. The four elastic small protrusions 20 form a group of protrusions 21. In the first embodiment, nine groups of the protrusions 21 are successively arranged in line in the region S1 in the longitudinal direction of the wide belt part 15. Thus 36 elastic small protrusions 20 are densely arranged in line in a region S2 having a width of 10 mm and a length of 0.90 mm so that when the brassiere 10 is worn, some of the elastic small protrusions 20 are placed at positions where they confront the kensei P1, the tenryou P2, and the kengaiyu P3 respectively.

Each of the elastic small protrusions 20 consists of silicone and has an approximately conic configuration having a height of 3 mm and a diameter of 3 mm at a bottom surface 20a thereof fixed to the wide belt part 15. The leading end of each elastic small protrusion 20 is circular arc-shaped. The difference between the height of the elastic small protrusion 20 when no load is applied thereto and the height thereof when a load of 50 g is applied thereto is set to less than 1 mm and 0.67 mm in the first embodiment.

The method of welding the elastic small protrusions 20 to the wide belt part 15 is carried out by casting curable silicone resin into a die for shaping the silicone resin into the small protrusion, bonding the inner surface of the wide belt part 15 to the die, and hardening the silicone resin in this state. In this manner, the elastic small protrusions 20 are mounted on the wide belt part 15.

Although the elastic small protrusions are made of the silicone in the first embodiment, the elastic small protrusions may be made of resin other than the silicone resin. Alternatively protrusions may be formed by intertwining threads with one another or by using a knitted structure or a woven structure.

The region S2 of the wide belt part 15 on which the elastic small protrusions 20 are mounted is covered with a covering cloth 16 consisting of a flexible mesh thin cloth. The covering cloth 16 is mounted on the shoulder strap body 14a of the shoulder strap 14 or the wide belt part 15 thereof by sewing the entire periphery of the covering cloth 16 thereto.

In the above-described construction, the wide belt part 15 is attached to the shoulder strap body 14a, with the wide belt part 15 protruded from the shoulder strap body 14a toward the neck, and a plurality of the elastic small protrusions 20 is densely mounted on the wide belt part 15. Therefore when the brassiere 10 is worn, it is possible to securely place one of the four elastic small protrusions 20 at positions where one elastic small protrusion confronts one of the kensei P1, the tenryou P2, and the kengaiyu P3. Thereby while the brassiere 10 is worn, it is possible to impart a weak tactile stimulus to the meridian points to such an extent that the elastic small protrusions 20 continually touch the meridian points. When a person wearing the brassiere 10 makes an ordinary motion of pressing the wide belt part 15 with her/his hand or lifting her/his shoulder, the elastic small protrusions 20 are temporarily pressed against the kensei P1, the tenryou P2, and the kengaiyu P3 at a little strong force, thus stimulating the meridian points.

Because the wide belt part 15 on which the elastic small protrusions 20 are mounted is made of the unstretchable material or the difficult-to-stretch material, the elastic small protrusions 20 are hardly dislocated from the predetermined positions respectively and can be securely positioned at the meridian points.

Because the elastic small protrusions 20 are densely arranged in the region S2 having the width of 10 mm and the length of 90 mm, the elastic small protrusions 20 can be disposed at the meridian points respectively irrespective of a person's physical constitution.

Further because the elastic small protrusions 20 are made of silicone, and the covering cloth 16 consisting of the thin cloth is interposed between the elastic small protrusions 20 and the person's skin to prevent the elastic small protrusions 20 from directly touching the person's skin, the elastic small protrusions 20 continually impart the tactile stimulus to the meridian points respectively while the person wears the brassiere 10. Therefore the elastic small protrusions 20 does not impart a feeling of physical disorder to the person nor a pain thereto, even though the person wears the brassiere 10 for a long time. The elastic small protrusions 20 are not strongly pressed against the meridian points but touch them lightly. But when the person lightly presses or moves her/his shoulder, the elastic small protrusions 20 are temporarily pressed against the meridian points, thus stimulating them a little strongly and making the person temporarily comfortable.

The above-described construction is applicable not only to the brassiere, but also to a brassiere-provided slip, lingerie, undershirt, sports clothes such as a swimming suit, leotard, and the like; and clothing for men having shoulder straps.

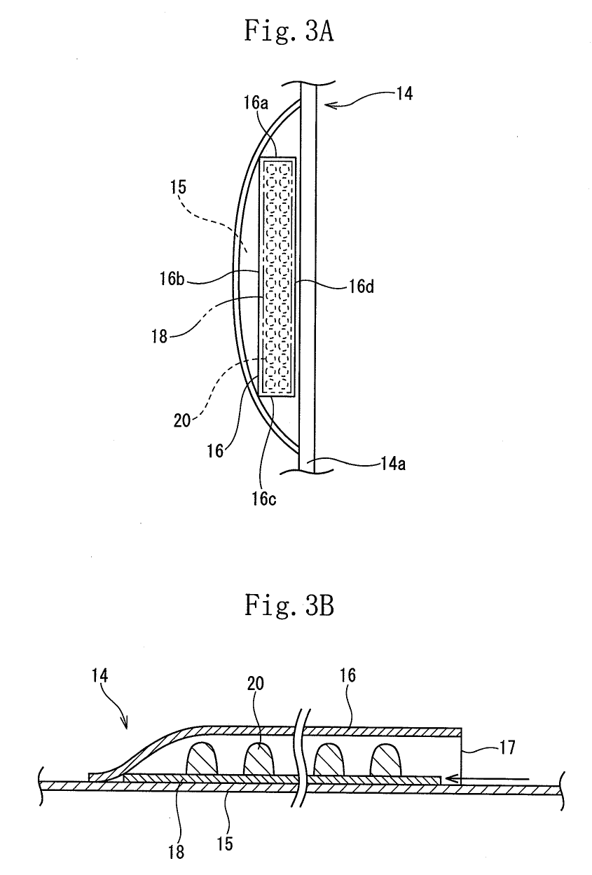

FIG. 3 shows a modification of the first embodiment.

In the brassiere of the modification, the elastic small protrusion 20 are mounted on a sheet-shaped base material 18 made of unstretchable synthetic leather. The base material 18 is not fixed to the clothing body but removably mounted thereon.

In detail, as shown in FIG. 3(A), the elastic rectangular covering cloth 16 is attached to the inner surface of the wide belt part 15 mounted on the shoulder strap 14 of the brassiere. Unlike the first embodiment, the entire periphery of the covering cloth 16 is not attached to the wide belt part 15, but except an upper-end shorter side 16a of the covering cloth 16 in FIG. 3(A), edges of other three sides 16b, 16c, and 16d thereof are attached to the wide belt part 15 by means of sewing or adhesion. Thereby the wide belt part 15 and the covering cloth 16 form a bag-shaped accommodating portion having an opening 17 formed at the side of the shorter side 16a. The opening 17 does not necessarily have to be formed at the shorter side of the covering cloth 16, but may be formed at the longer side thereof.

Unlike the first embodiment, the elastic small protrusions 20 are mounted on the wide belt part 15 not directly but on the base material 18 provided separately from the clothing body, as described above. The size of the base material 18 is set a little smaller than that of the covering cloth 16. The elastic small protrusions 20 are mounted on the base material 18 in the same arrangement as that of the first embodiment.

As shown in FIG. 3(B), with the surface of the base material 18 on which the elastic small protrusions 20 are mounted in confrontation with the covering cloth 16, the base material 18 is inserted into the accommodating space surrounded with the bag-shaped wide belt part 15 and the covering cloth 16 through the opening 17. When the elastic small protrusions 20 are not required, the base material 18 can be pulled out of the accommodating space through the opening 17.

In the above-described construction, the elastic small protrusions 20 can be mounted on the brassiere at a necessary position thereof and at only a necessary time. In addition, it is possible to arbitrarily select the height, hardness, and configuration of the elastic small protrusions 20. Thus it is possible to use the brassiere in a mode suitable for the person's demand.

By mounting the base material 18 to which the elastic small protrusions 20 have been attached on clothing other than the brassiere, the base material can be also used other clothing.

By removing the base material from the clothing body when the brassiere is washed, it is possible to prevent the elastic small protrusions from being broken and peeling off the base material.

The construction of the modification is applicable to the other embodiments which are described later. Other constructions of the modification and the operation and effect thereof are similar to those of the first embodiment. Thus the same parts of the modification as those of the first embodiment are denoted by the same reference numerals as those of the first embodiment, and description thereof is omitted herein.

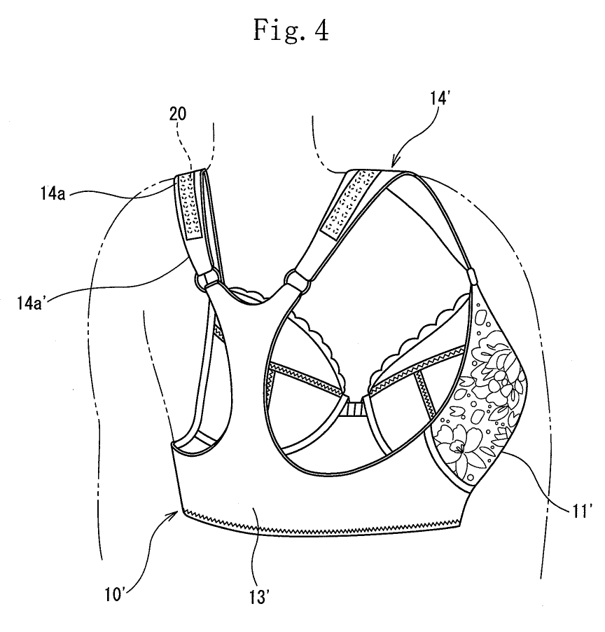

FIG. 4 shows the second embodiment.

As shown in FIG. 4, the configuration of a back part 13′ and that of a shoulder strap 14′ of the brassiere 10′ of the second embodiment are different from that of the back part 13 and that of the shoulder strap 14 of the first embodiment respectively. That is, the back part 13′ is extended upward along the line of the backbone, is branched to a left-hand shoulder strap body 14a′ and a right-hand shoulder strap body 14a′ at an upper side of the back part 13′, and a leading end of the shoulder strap body 14a′ is mounted at an upper end of the cup part 11′. By configuring the shoulder strap 14 in such a way that the left and right shoulder strap body 14a′ are near to each other at the cup part 11′ and the back part 13′, the shoulder strap body 14a′ is located nearer toward the neck than the shoulder straps of ordinary brassieres so that the shoulder strap body 14a′ covers the meridian points disposed in the upper region of the shoulders.

The shoulder strap body 14a′ is formed widely in the upper region of the shoulders, and the widened part of the shoulder strap body 14a′ is made of an unstretchable material or a difficult-to-stretch material. Alternatively the unstretchable material or the difficult-to-stretch material is mounted on the wide part of the shoulder strap body 14a′. Similarly to the first embodiment, four elastic small protrusions 20 are provided in a region having an area of 100 square millimeters to form a group of protrusions 21. Nine groups of the protrusions 21 are successively arranged in line on the shoulder strap body 14a′ in the longitudinal direction thereof.

In the above-described construction, the shoulder strap body 14a′ of the brassiere 10′ is disposed nearer to the neck than shoulder strap bodies of ordinary brassieres in the upper region of the shoulders. Thus it is not necessary to provide the shoulder strap body 14a′ with a large wide part, but it is only necessary to provide the shoulder strap body 14a′ with the wide part having a width to such an extent that a necessary number of the elastic small protrusions 20 to be disposed at the meridian points P1, P2, and P3 is mounted on the shoulder strap body 14a′. Thereby similarly to the first embodiment, the elastic small protrusions 20 can be securely placed at positions where the elastic small protrusions 20 confront the meridian points and are capable of imparting a weak tactile stimulus to the meridian points with the elastic small protrusions 20 in continual contact with the meridian points at a weak force, while the brassiere 10 is worn. When the person wearing the brassiere 10′ presses the wide part of the shoulder strap body 14a′ with the person's hand or moves the person's body, the elastic small protrusions 20 are temporarily pressed against the meridian points respectively at a little strong force. Thus the elastic small protrusions 20 are capable of temporarily imparting a comfortable feeling to the person.

Other constructions of the second embodiment and the operation and effect thereof are similar to those of the first embodiment. Thus the same parts of the second embodiment as those of the first embodiment are denoted by the same reference numerals as those of the first embodiment, and description thereof is omitted herein.

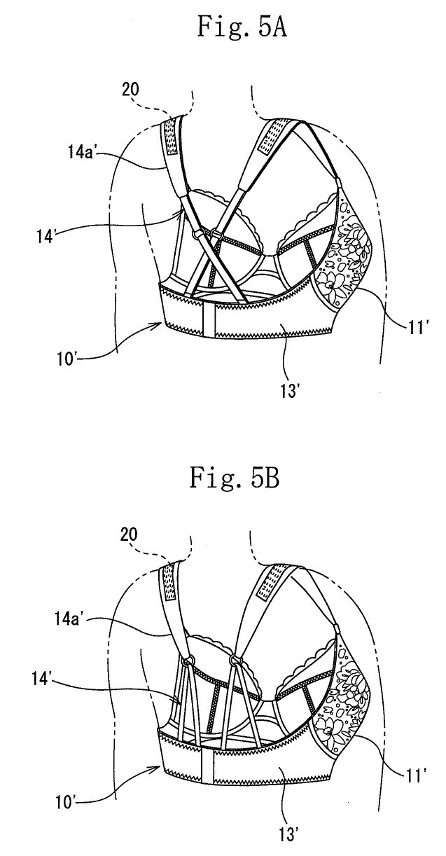

FIG. 5 shows modifications of the second embodiment.

In the modification shown in FIG. 5(A), the shoulder strap bodies 14a′ cross each other at the back side.

In the modification shown in FIG. 5(B), each of the left and right shoulder strap bodies 14a′ is bifurcated on the back side. The bifurcated shoulder strap bodies 14a′ are mounted on the back part 13′. Similarly to the second embodiment, the shoulder strap body 14a′ is provided with the elastic small protrusions 20.

The above-described construction allows the shoulder strap body 14a′ to be located nearer toward the neck than the shoulder straps of ordinary brassieres so that the shoulder strap 14′ covers the portion where the stiffness of the shoulders is generated and allows the elastic small protrusions 20 to be placed at the meridian points respectively.

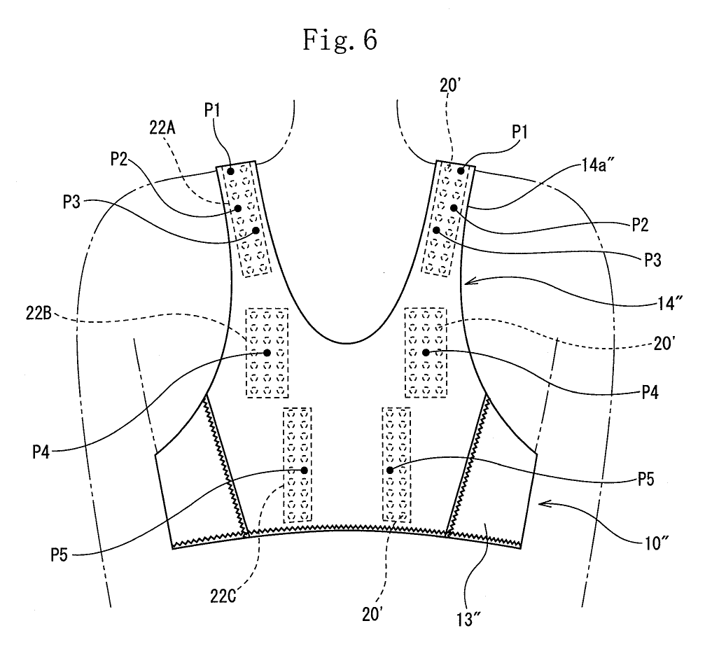

FIG. 6 shows a third embodiment.

A brassiere 10″ of the third embodiment is always capable of imparting a slight tactile stimulus not only to the kensei P1, the tenryou P2, and the kengaiyu P3, but also to the koukou P4 and the kanyu P5 disposed below the kensei P1, the tenryou P2, and the kengaiyu P3. Shoulders are also liable to grow stiff in the neighborhood of the koukou P4 and the kanyu P5.

In the brassiere 10″, a back part 13″ and a shoulder strap 14″ are continuously formed to widely cover the back of the person wearing the brassiere 10″ as shown in FIG. 6. Similarly to the second embodiment, the shoulder strap body 14a″ is located nearer toward the neck than the shoulder straps of ordinary brassieres so that the shoulder strap body 14a″ covers the meridian points disposed in the upper region of the shoulders. The configuration of the brassiere 10″ on the back side of the brassiere 10″ is not limited to that shown in FIG. 6, but the brassiere 10″ may have any desired configurations, provided that the brassiere 10″ covers the person's kensei P1, the tenryou P2, the kengaiyu P3, the koukou P4, and the kanyu P5.

In the third embodiment, the inner surface of the shoulder strap 14″ and that of the back part 13″ are provided with a first unstretchable material 22A covering the kensei P1, the tenryou P2, and the kengaiyu P3; a second unstretchable material 22B covering the koukou P4; and a third unstretchable material 22C covering the kanyu P5. The difficult-to-stretch material may be used instead of the unstretchable material.

Semispherical elastic small protrusions 20′ having a diameter of 3 mm are fixed to the first unstretchable material 22A through the third unstretchable material 22C. That is, in the third embodiment, each of the unstretchable materials 22A through 22C is made of a material having a high tension, and the height of each of the elastic small protrusions 20′ is set smaller than that of the elastic small protrusion of the first embodiment.

The elastic small protrusions disposed on the material having a high tension are capable of constantly imparting the tactile stimulus to the meridian points respectively to an extent similar to the extent of the pressing force of taller elastic small protrusions disposed on a material having a low tension and further temporarily stimulating the meridian points respectively at a little strong pressing force when the person makes a motion.

In a manner similar to that of the first embodiment, the elastic small protrusions 20′ are arranged on the first unstretchable material 22A having an area of 50 to 120 mm² covering the periphery of the person's kensei P1, tenryou P2, and kengaiyu P3.

The second unstretchable material 22B covering the periphery of the koukou P4 is provided with three elastic small protrusions 20′ arranged widthwise and seven elastic small protrusions 20′ arranged longitudinally. Thus in total, 21 elastic small protrusions 20′ are mounted on the second unstretchable material 22B. The interval between the adjacent elastic small protrusions 20′ is set equally to the interval between the adjacent elastic small protrusions 20′ of the first unstretchable material 22A.

Four elastic small protrusions 20′ are mounted on the third unstretchable material 22C covering the periphery of the kanyu P5 at a 10-millimeter square region of the inner surface thereof, namely, the region having an area of 100 mm². The four elastic small protrusions 20′ form one group of protrusions. Five groups of the protrusions are vertically and successively arranged in line.

In the above-described construction, when the brassiere 10′ is worn, it is possible to place the elastic small protrusions 20′ at positions where the elastic small protrusions 20′ confront the kensei P1, tenryou P2, kengaiyu P3, koukou P4, and kanyu P5 softening the stiffness of the shoulders respectively. Thereby while the brassiere 10 is worn, the elastic small protrusions 20′ contact the meridian points to such an extent that the elastic small protrusions 20′ continually touch the meridian points, thus imparting the tactile stimulus to the meridian points. When the person wearing the brassiere 10 makes touches the elastic small protrusions 20′ lightly or moves the person's body, the elastic small protrusions 20′ are temporarily pressed against not only the kensei P1, the tenryou P2, and the kengaiyu P3, but also the koukou P4 and kanyu P5 at a comparatively strong force, thus imparting a little strong stimulus to the meridian points and making the person feel temporarily comfortable.

Other constructions of the third embodiment and the operation and effect thereof are similar to those of the first embodiment. Thus the same parts of the third embodiment as those of the first embodiment are denoted by the same reference numerals as those of the first embodiment, and description thereof is omitted herein.

The upper side of the shoulder strap of the brassiere of the third embodiment is formed in a small width in such a way that the shoulder strap passes only the neighborhood of the kensei P1, the tenryou P2, and the kengaiyu P3. But when the shoulder strap is wide, the first unstretchable material on which the elastic small protrusions are mounted is also provided at the neck side where the kensei P1, the tenryou P2, and the kengaiyu P3 are positioned so that the elastic small protrusions contact the predetermined meridian points.

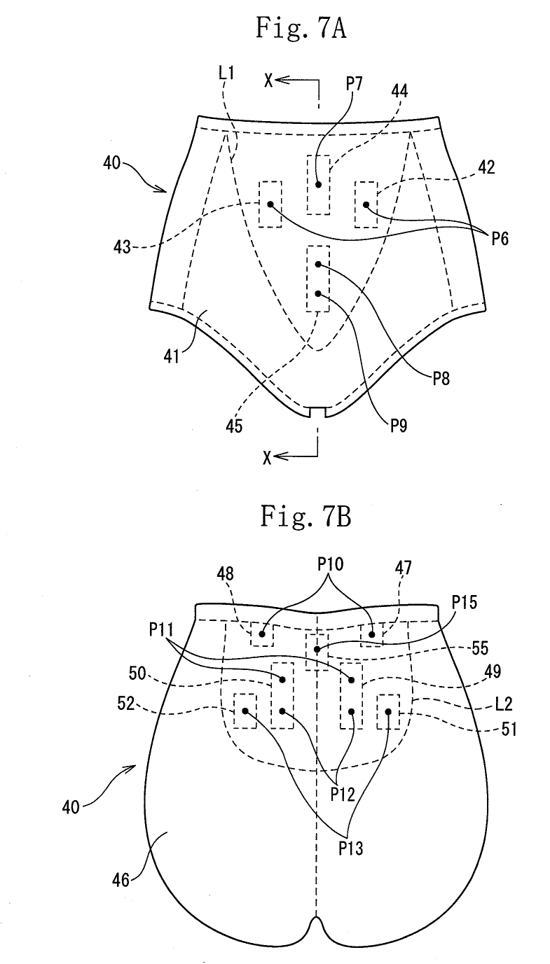

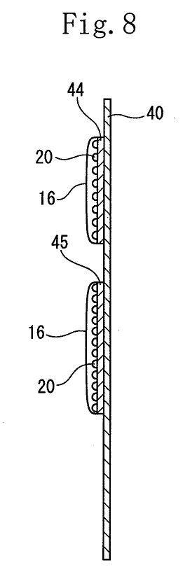

FIGS. 7 and 8 show a girdle 40 of a fourth embodiment for protecting the person from cold.

Unstretchable materials to which elastic small protrusions are densely fixed are mounted on an inner surface of the girdle 40 at positions thereof covering the meridian points.

In detail, mounted in the front panel 41 covering the belly are unstretchable materials 42, 43, having a length of 3 cm×a width of 1 cm, which are placed at positions covering left and right daikyo P6; an unstretchable material 44, having a length of 4 cm×a width of 1 cm, which is placed at a position covering a kikai P7; and an unstretchable material 45, having a length of 5 cm×a width of 1 cm, which is placed at a position covering the kangen P8 and the chukyoku P9.

Mounted in a back panel 46 covering the body in the range from the waist to the buttocks are unstretchable materials 47, 48, having a length of 1 cm×a width of 1 cm, which are placed at positions covering the left and right daichoyu P10; unstretchable materials 49, 50, having a length of 5 cm×a width of 1 cm, which are placed at positions covering the left and right jiryou P11 and the left and right churyou P12; unstretchable materials 51, 52, having a length of 2 cm×a width of 1 cm, which are placed at positions covering the left and right houkou P13; unstretchable materials 53, 54 having a length of 1 cm×a width of 1 cm, which are placed at positions covering the left and right jinyu P14; and an unstretchable material 55, having a length of 3 cm×a width of 1 cm, which is placed at a position covering the jousen P15.

On the above-described unstretchable material, four elastic small protrusions are disposed per 1 cm². Because the width of the unstretchable material is set to 1 cm, two elastic small protrusions are arranged in the left-to-right direction.

All of the above-described unstretchable materials used in the fourth embodiment are similar to that of the first embodiment. The elastic small protrusions 20 fixed to the unstretchable materials respectively are formed in a manner similar to that of the first embodiment.

Except the elastic small protrusions on the unstretchable materials 49, 50 placed at the positions covering the left and right jiryou P11 and the left and right churyou P12, the heights of all of the other elastic small protrusions are set to 2 mm. The heights of the elastic small protrusions mounted on the unstretchable materials 49, 50 are set to 3 mm.

Every elastic small protrusion 20 is semispherical. The diameter of the area of the bottom of the elastic small protrusion 20 is set to 3 mm. The thin covering cloth 16 is mounted on each of the unstretchable materials to cover the surfaces of the elastic small protrusions 20.

When the girdle 40 is worn without wearing shorts or when the girdle 40 is worn on the shorts, any one of the elastic small protrusions continually impart a light tactile stimulus to one of the meridian points with the elastic small protrusions placed at positions where they confront the meridian points respectively. When the belly or the waist is pressed with the hand of the person wearing the girdle 40 or/and the shorts or when the person moves the body thereof, the elastic small protrusions contact the meridian points at a little strong pressing force, thus making the person feel temporarily comfortable. As described above, the elastic small protrusions continually impart the tactile stimulus to the meridian points capable of softening the cold at the belly and the waist, the menorrhalgia, and the menorrhalgia. Further when the meridian points are stimulated by pressing the belly and the waist with the person's hand or by temporarily pressing the meridian points at a little strong force by a person's simple motion, it is possible to accelerate the circulation of blood. Consequently the girdle 40 can be used to soften the cold, the menorrhalgia, and the lumbago.

The mounting positions of the unstretchable materials 42 through 45 at the belly side are located in a region surrounded with a one-dot chain line L1 in an ordinary girdle. Thus the elastic small protrusions may be directly fixed to one unstretchable material composing the girdle without individually mounting the unstretchable materials on the girdle.

At the waist side, one unstretchable material may be mounted in a region, surrounded with a one-dot chain line L2, in which the unstretchable materials 47 through 52 are provided. The elastic small protrusions may be directly fixed to the unstretchable material without individually mounting the unstretchable materials on the girdle.

Monitors wore the girdle 40 of the present invention for 20 minutes to measure the circulation of blood. As a result, there was a tendency for the circulation of blood to be enhanced. There was a difference between the result obtained when they wore the girdle 40 of the present invention and the result obtained when they wore ordinary girdles. All of monitors who are oversensitive to cold actually felt that the girdle had the effect of decreasing the degree of the cold. Further the elastic small protrusion did not make the monitors feel uncomfortable.

1. Clothing comprising a plurality of elastic small protrusions, each having a diameter of 2 to 4 mm at a bottom face thereof, which are provided on an inner surface thereof at positions covering meridian points of a human body wearing said clothing, with said elastic small protrusions disposed in a region of 50 to 120 mm for one of said meridian points so that even if a positional shift of said clothing takes place and irrespective of a physical constitution of said human body wearing said clothing, any one of the elastic small protrusions is disposed at a position where said elastic small protrusion confronts one of said meridian points and imparts a tactile stimulus to said meridian point.

2. The clothing according to claim 1, wherein a number of said elastic small protrusions provided inside said region having said area of 50 mm² to 120 mm² is set to four, spaced at intervals of 90 degrees; and said elastic small protrusions are disposed regularly, with said four elastic small protrusions forming one group.

3. The clothing according to claim 1, wherein a height of said elastic small protrusions is set to 2 to 4 mm.

4. The clothing according to claim 3, wherein when a base material on which said elastic small protrusions are provided is made of a material having a high tension, said height of said elastic small protrusions is set small in said range, whereas when said base material on which said elastic small protrusions are provided is made of a material having a low tension, said height of said elastic small protrusions is set large in said range.

5. The clothing according to claim 1, wherein said elastic small protrusions are made of resin or fiber; and a difference between said height of said elastic small protrusions when no load is applied thereto and said height thereof when a load of 50 g is applied thereto is set to less than 1 mm; and
each of said elastic small protrusions is semispherical, spherical or a conic configuration, a leading portion of which is formed as a circular-arc surface.

6. The clothing according to claim 1, wherein said base material on which said elastic small protrusions are provided is made of an unstretchable material or a difficult-to-stretch material; said base material is fixedly mounted on said clothing body at a necessary position thereof or removably mounted on a holding portion provided on said clothing body at a necessary position thereof, with said elastic small protrusions protruded toward the skin of said human body.

7. The clothing according to claim 1, wherein said region in which said elastic small protrusions are provided is covered with a covering cloth consisting of thin cloth so that said elastic small protrusions are brought into contact with said skin of said human body through said covering cloth.

8. The clothing according to claim 1, wherein a wide base material made of an unstretchable material or a difficult-to-stretch material is attached to said left and right shoulder strap bodies mounted on said clothing from said front panel thereof to said back panel thereof at positions thereof essentially covering said meridian points disposed on a periphery of an upper region of a shoulder, with said wide base material protruded toward the neck of the human body; and said wide base material is provided with said elastic small protrusions located at positions of an inner surface thereof covering said meridian points.

9. The clothing according to claim 1, wherein said left and right shoulder strap bodies mounted on said clothing from said front panel thereof to said back panel thereof are connected with each other with said left and right shoulder strap bodies disposed in proximity to each other at a front panel side and a back panel side; said left and right shoulder strap bodies are brought near to the neck in an upper region of a shoulder so that said left and right shoulder strap bodies cover said meridian points; and each of said left and right shoulder strap bodies is widened in said upper region of said shoulder and made of a unstretchable material or a difficult-to-stretch material; and
wherein each of said left and right shoulder strap bodies widened in said upper region of said shoulder is provided with said elastic small protrusions by locating said elastic small protrusions at meridian point-covering positions of an inner surface of said left and right shoulder strap bodies.

10. The clothing according to claim 1, wherein said elastic small protrusions are provided at positions covering meridian points positioned at the back, the belly or/and the waist.

11. The clothing according to claim 1, comprising underwear such as a brassiere, a brassiere-provided slip, lingerie, undershirt, girdle, shorts, pants; and sports clothing such as a swimming suit, leotard, and the like.

12. The clothing according to claim 2, wherein a height of said elastic small protrusions is set to 2 to 4 mm.

13. The clothing according to claim 12, wherein when a base material on which said elastic small protrusions are provided is made of a material having a high tension, said height of said elastic small protrusions is set small in said range, whereas when said base material on which said elastic small protrusions are provided is made of a material having a low tension, said height of said elastic small protrusions is set large in said range.
